(19) United States
(12) Patent Application Publication
(10) Pub. No.: US 2015/0164659 A1
(43) Pub. Date: Jun. 18, 2015

(54) STRUCTURE FOR LINKING UPPER PROSTHETIC LEG PART AND LOWER PROSTHETIC LEG PART IN PROSTHETIC LEG

(71) Applicant: Yukio KONISHI, Nagaoka-shi, Niigata (JP)

(72) Inventor: Yukio Konishi, Nagaoka-shi (JP)

(21) Appl. No.: 14/414,805

(22) PCT Filed: Jul. 26, 2013
(86) PCT No.: PCT/JP2013/070281

§ 371 Date: Jan. 14, 2015

(30) Foreign Application Priority Data
Jul. 31, 2012 (JP) 2012-170550
Jul. 22, 2013 (JP) 2013-151572

(51) Int. Cl. A61F 2/60

(52) U.S. Cl. CPC A61F 2/60; A61F 2002/607

(57) ABSTRACT

The purpose of the present invention is to provide a structure for linking an upper prosthetic leg part and a lower prosthetic leg part in a revolutionary prosthetic leg affording advantageous working effects not obtained in the prior art. A linking structure for an upper prosthetic leg part 1 fitted to the body, and a lower prosthetic leg part 2 linked to this upper prosthetic leg part 1 to constitute a prosthetic leg F, a ground-contacting part 2a being provided at a lower end; wherein the structure is constituted by a linking part 3 provided to either the lower prosthetic leg part 2 or the upper prosthetic leg part 1, and a mated linking part 4 provided to the other and adapted to mate with the linking part 3; and having retaining elements 6, 7 which protrude into the mating aperture 4a of the mated linking part 4 and retain the linking part 3 positioned within the mating aperture 4a.

### CROSS REFERENCE TO RELATED APPLICATIONS

This application is a National Stage of International Application No. PCT/JP2013/070281 filed Jul. 26, 2013, claiming priority based on Japanese Patent Application Nos. 2012-170550 filed Jul. 31, 2012 and 2013-151572 filed Jul. 22, 2013, the contents of all of which are incorporated herein by reference in their entirety.

### TECHNICAL FIELD

The present invention relates to a structure for linking an upper prosthetic leg part and a lower prosthetic leg part in a prosthetic leg.

### BACKGROUND ART

As a prosthetic leg intended to replace a leg lost in an accident or the like, there have been proposed a prosthetic leg (femoral prosthetic leg) comprising an upper prosthetic leg part (socket part) which is fitted to the body (a femoral stump of a severed leg), and a lower prosthetic leg part (lower leg prosthetic limb part) linked to this upper prosthetic leg part and having a ground-contacting part (foot part) at the lower end. The present applicant, in Japanese Patent Publication 4741635, previously proposed a prosthetic leg (“prior art example”). The lower prosthetic leg part is equipped with a flexing part that functions as a knee joint, making flexing motion possible.

The prior art example is a prosthetic leg comprising an upper prosthetic leg part fitted to the body, and a lower prosthetic leg part linked to this upper prosthetic leg part and having a ground-contacting part at the lower end, the lower prosthetic leg part being equipped with a protruding part, and the upper prosthetic leg part being equipped with a mated linking part. The prior art example is constituted such that a ring member mates with this mated linking part, the thickness of the linking structure of the upper prosthetic leg part and the lower prosthetic leg part constituting a portion of the thickness of the mated linking part. The protruding part is placed within a ring aperture of the ring member, the ring member being equipped with a retaining element that protrudes into the ring aperture. The protruding part positioned within the ring aperture is retained while gripped by this retaining element, and the retaining element is retained by the proximal end of the retaining element bearing on the mated linking part.

This constitution affords the ability to obtain a state of optimal fit permitting satisfactory ambulation, and has certain advantageous working effects, such as the absence of any concern regarding poor balance of the left and right foot despite the considerable overall length and the absence of any incidence of change in the adjusted angle due to repeated use, while enabling consistently satisfactory ambulation.

### PRIOR ART DOCUMENT

### Patent Document

[Patent Document 1] Japanese Patent Publication 4741635

### DISCLOSURE OF THE INVENTION

### Problems the Invention is Intended to Solve

As a result of additional research regarding a prosthetic leg, the applicant developed a structure for linking an upper prosthetic leg part and a lower prosthetic leg part in a revolutionary prosthetic leg which affords advantageous working effects not obtained in the prior art.

### Means for Solving the Problems

The main points of the present invention are described below with reference to the accompanying drawings.

The present invention relates to a structure for linking an upper prosthetic leg part 1 fitted to the body, and a lower prosthetic leg part 2 linked to the upper prosthetic leg part 1 to constitute a prosthetic leg F, a ground-contacting part 2a being provided at a lower end, the structure for linking an upper prosthetic leg part and a lower prosthetic leg part in a prosthetic leg being characterized in being constituted by a linking part 3 provided to either the lower prosthetic leg part 2 or the upper prosthetic leg part 1, and a mated linking part 4 provided to the other of the lower prosthetic leg part 2 and the upper prosthetic leg part 1 and adapted to mate with the linking part 3; there being provided retaining elements 6, 7 which protrude into the mating aperture 4a of the mated linking part 4 and retain the linking part 3 positioned within the mating aperture 4a, the retaining elements 6, 7 being constituted by a first retaining element 6 which protrudes into the mating aperture 4a in a direction orthogonal to the axis of the mated linking part 4 and retains the linking part 3, and a second retaining element 7 which protrudes into the mating aperture 4a in a direction diagonal to the axis of the mated linking part 4 and retains the linking part 3; the degree of protrusion of the first retaining element 6 into the mating aperture 4a being variable, and the linking part 3 being moveably linked within a plane orthogonal to the axis of the mated linking part 4 within the mating aperture 4a.

The present invention relates also to the structure for linking an upper prosthetic leg part and a lower prosthetic leg part in a prosthetic leg according to the first aspect, wherein the structure for linking an upper prosthetic leg part and a lower prosthetic leg part in a prosthetic leg is characterized in that the degree of protrusion of the second retaining element 7 into the mating aperture 4a is variable, and the linking part 3 is tiltably linked within the mating aperture 4a.

The present invention relates also to the structure for linking an upper prosthetic leg part and a lower prosthetic leg part in a prosthetic leg according to the first aspect, wherein the structure for linking an upper prosthetic leg part and a lower prosthetic leg part in a prosthetic leg is characterized in that the mated linking part 4 is provided with a plurality of through-holes 8, the first retaining element 6 and the second retaining element 7 being inserted into each of the through-holes 8.

The present invention relates also to the structure for linking an upper prosthetic leg part and a lower prosthetic leg part in a prosthetic leg according to the second aspect, wherein the structure for linking an upper prosthetic leg part and a lower prosthetic leg part in a prosthetic leg is characterized in that the mated linking part 4 is provided with a plurality of through-holes 8, the first retaining element 6 and the second retaining element 7 being inserted into each of the through-holes 8.

The present invention relates also to the structure for linking an upper prosthetic leg part and a lower prosthetic leg part in a prosthetic leg according to the third aspect, wherein the structure for linking an upper prosthetic leg part and a lower prosthetic leg part in a prosthetic leg is characterized in that the through-hole 8 into which the second retaining element 7 inserts is provided obliquely relative to the axis of the mated linking part 4.

The present invention relates also to the structure for linking an upper prosthetic leg part and a lower prosthetic leg part in a prosthetic leg according to the fourth aspect, wherein the structure for linking an upper prosthetic leg part and a lower prosthetic leg part in a prosthetic leg is characterized in that the through-hole 8 into which the second retaining element 7 inserts is provided obliquely relative to the axis of the mated linking part 4.

The present invention relates also to the structure for linking an upper prosthetic leg part and a lower prosthetic leg part in a prosthetic leg according to any of the first through sixth aspects, wherein the structure for linking an upper prosthetic leg part and a lower prosthetic leg part in a prosthetic leg is characterized in that an outside peripheral surface 3a of the linking part 3 defines an inclined surface that widens to the distal end side.

The present invention relates also to a structure for linking an upper prosthetic leg part 1 fitted to the body, and a lower prosthetic leg part 2 linked to the upper prosthetic leg part 1 to constitute a prosthetic leg F, a ground-contacting part 2a being provided at a lower end, the structure for linking an upper prosthetic leg part and a lower prosthetic leg part in a prosthetic leg being characterized in being constituted by a linking part 3 provided to either the lower prosthetic leg part 2 or the upper prosthetic leg part 1, and a mated linking part 4 provided to the other of the lower prosthetic leg part 2 and the upper prosthetic leg part 1 and adapted to mate with the linking part 3; there being provided retaining elements 6, 7 which protrude into the mating aperture 4a of the mated linking part 4 and retain the linking part 3 positioned within the mating aperture 4a, the retaining elements 6, 7 being constituted by a first retaining element 6 which protrudes into the mating aperture 4a in a direction orthogonal to the axis of the mated linking part 4 and retains the linking part 3, and a second retaining element 7 which protrudes into the mating aperture 4a obliquely relative to the axis of the mated linking part 4 and retains the linking part 3; the mated linking part 4 being provided with a plurality of through-holes 8, and the first retaining element 6 and the second retaining element 7 being inserted into each of the through-holes 8.

The present invention relates also to the structure for linking an upper prosthetic leg part and a lower prosthetic leg part in a prosthetic leg according to the eighth aspect, wherein the structure for linking an upper prosthetic leg part and a lower prosthetic leg part in a prosthetic leg is characterized in that the through-hole 8 into which the second retaining element 7 inserts is provided obliquely relative to the axis of the mated linking part 4.

The present invention relates also to the structure for linking an upper prosthetic leg part and a lower prosthetic leg part in a prosthetic leg according to the eighth or ninth aspect, wherein the structure for linking an upper prosthetic leg part and a lower prosthetic leg part in a prosthetic leg is characterized in that the degree of protrusion of the second retaining element 7 into the mating aperture 4a is variable, and the linking part 3 is tiltably linked within the mating aperture 4a.

The present invention relates also to a structure for linking an upper prosthetic leg part 1 fitted to the body, and a lower prosthetic leg part 2 linked to the upper prosthetic leg part 1 to constitute a prosthetic leg F, a ground-contacting part 2a being provided at a lower end, the structure for linking an upper prosthetic leg part and a lower prosthetic leg part in a prosthetic leg being characterized in being constituted by a linking part 3 provided to either the lower prosthetic leg part 2 or the upper prosthetic leg part 1, and a mated linking part 4 provided to the other of the lower prosthetic leg part 2 and the upper prosthetic leg part 1 and adapted to mate with the linking part 3; there being provided retaining elements 6, 7 which protrude into the mating aperture 4a of the mated linking part 4 and retain the linking part 3 positioned within the mating aperture 4a, the retaining elements 6, 7 being constituted by a first retaining element 6 which protrudes into the mating aperture 4a in a direction orthogonal to the axis of the mated linking part 4 and retains the linking part 3, and a second retaining element 7 which protrudes into the mating aperture 4a obliquely relative to the axis of the mated linking part 4 and retains the linking part 3; and an outside peripheral surface 3a of the linking part 3 defines an inclined surface that widens to the distal end side.

### Effect of the Invention

The present invention, due to the constitution set forth above, provides a revolutionary structure for linking an upper prosthetic leg part and a lower prosthetic leg part in a prosthetic leg, which affords the ability to obtain an state of optimal fit permitting satisfactory ambulation, and which moreover has advantageous working effects not encountered in the prior art, such as the ability to easily and rapidly make adjustments required by the user according to circumstances.

### BRIEF DESCRIPTION OF THE DRAWINGS

FIG. 1 is a descriptive diagram of a state of usage according to a first embodiment;

FIG. 2 is an exploded perspective view of a relevant part of the first embodiment;

FIG. 3 is a cross sectional view of a relevant part of the first embodiment;

FIG. 4 is a cross sectional view describing operation of a relevant part of the first embodiment;

FIG. 5 is a cross sectional view describing operation of a relevant part of the first embodiment;

FIG. 6 is a cross sectional view describing operation of a relevant part of the first embodiment;

FIG. 7 is an exploded perspective view of another example of the first embodiment;

FIG. 8 is a cross sectional view of another example of the first embodiment;

FIG. 9 is a descriptive diagram of a state of usage according to a second embodiment;

FIG. 10 is an exploded perspective view of a relevant part of the second embodiment;

FIG. 11 is a cross sectional view of a relevant part of the second embodiment;

FIG. 12 is a cross sectional view of a relevant part of the second embodiment;

FIG. 13 is a cross sectional view describing operation of a relevant part of the second embodiment;

FIG. 14 is a cross sectional view describing operation of a relevant part of the second embodiment;

FIG. 15 is a cross sectional view describing operation of a relevant part of the second embodiment;

FIG. 16 is a cross sectional view describing operation of a relevant part of a third embodiment; and

FIG. 17 is a cross sectional view describing operation of a relevant part of the third embodiment.

### BEST MODE FOR CARRYING OUT THE INVENTION

The preferred modes of the present invention will be described briefly on the basis of the accompanying drawings, to show the effects of the present invention.

In a case in which the upper prosthetic leg part 1 and the lower prosthetic leg part 2 are to be linked, the linking part 3 which is provided to either the lower prosthetic leg part 2 or the upper prosthetic leg part 1, and the mated linking part 4 provided to the other of the lower prosthetic leg part 2 and the upper prosthetic leg part 1, are mated, and the linking part 3 is retained within the mating aperture 4a by the retaining elements 6, 7 which protrude into the mating aperture 4a of the mated linking part 4.

The retaining elements 6, 7 are made to protrude to an appropriate extent and retain the linking part 3 while the linking part 3 is retained by the retaining elements 6, 7, so that an optimal state of fit with respect to the body is achieved (the upper prosthetic leg part 1 and the lower prosthetic leg part 2 are linked at an optimal angle).

In specific terms, the retaining elements 6, 7 are constituted by the first retaining element 6 which protrudes into the mating aperture 4a in a direction orthogonal to the axis of the mated linking part 4 and retains the linking part 3, and the second retaining element 7 which protrudes into the mating aperture 4a in a direction diagonal to the axis of the mated linking part 4 and retains the linking part 3.

Due to this constitution, by appropriately varying the degree of protrusion of the first retaining element 6 into the mating aperture 4a, the first retaining element 6 protrudes in a direction orthogonal to the axis of the mated linking part 4 and presses the linking part 3; therefore, the linking part 3 moves, in this case in a horizontal direction, within a plane orthogonal to the axis of the mated linking part 4 within the mating aperture 4a. Meanwhile, by appropriately varying the degree of protrusion of the second retaining element 7 into the mating aperture 4a, the second retaining element 7 protrudes obliquely relative to the axis of the mated linking part 4 within the mating aperture 4a and presses the linking part 3; therefore, the linking part 3 tilts within the mating aperture 4a.

That is, for example, position adjustment of the lower prosthetic leg part 2 in a horizontal direction (e.g., the front-back direction or left-right direction) with respect to the upper prosthetic leg part 1 may be satisfactorily carried out merely by adjusting the degree of protrusion of the first retaining element 6, while angular adjustment of the lower prosthetic leg part 2 with respect to the upper prosthetic leg part 1 may be satisfactorily carried out merely by adjusting the degree of protrusion of the second retaining element 7.

Consequently, a state of optimal fit permitting satisfactory ambulation can be obtained easily, and adjustments required by the user according to circumstances can be made easily and rapidly.

### Example 1

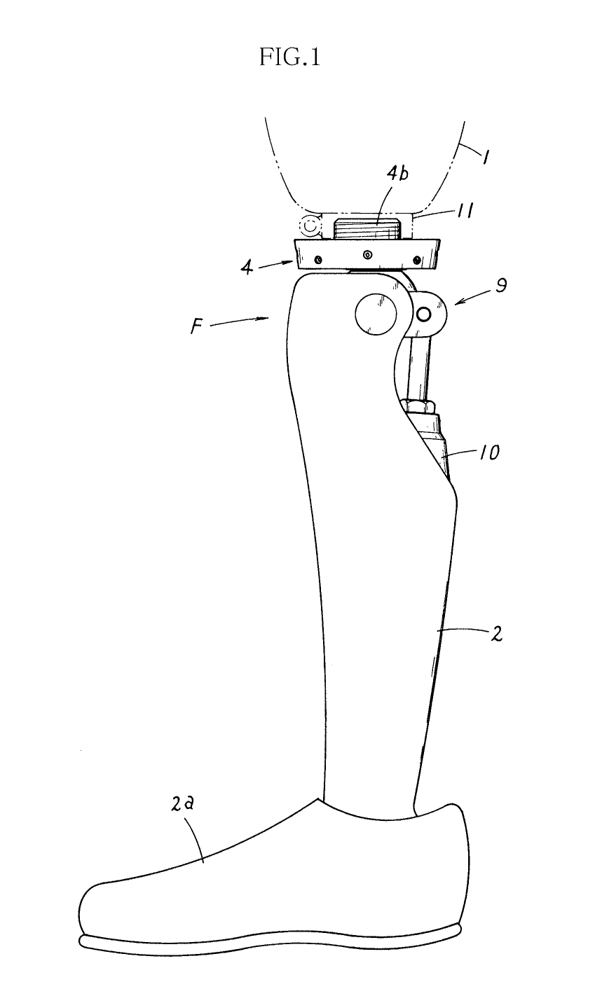

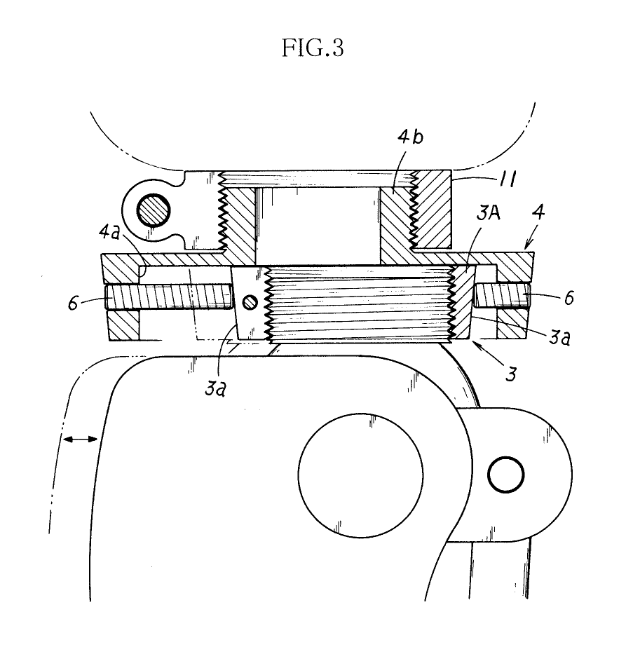

A specific first example of the present invention will be described on the basis of FIGS. 1 to 8.

The present example is a prosthetic leg (femoral prosthetic leg) composed of the upper prosthetic leg part 1 (socket part) which is fitted to the body (a femoral stump of a severed leg), and the lower prosthetic leg part 2 (lower leg prosthetic limb part) which is linked to the upper prosthetic leg part 1 and has a ground-contacting part 2a (foot part) at the lower end. The upper part of the lower prosthetic leg part is provided with a flexing part 9 of widely-known structure, which functions as a knee joint, making flexing motion possible; however, a structure lacking the flexing part 9 would also be acceptable. Drawing symbol 10 indicates a cylinder device.

In the present example, the upper prosthetic leg part 1 and the lower prosthetic leg part 2 are linked via a linking structure equipped with an angular adjustment function.

In specific terms, the linking structure is constituted by the linking part 3 provided to the upper part of the lower prosthetic leg part 2, and the mated linking part 4 which is matingly linked to the linking part 3. It is also acceptable to provide the linking part 3 to the upper prosthetic leg part 1, while providing the mated linking part 4 to the lower prosthetic leg part 2.

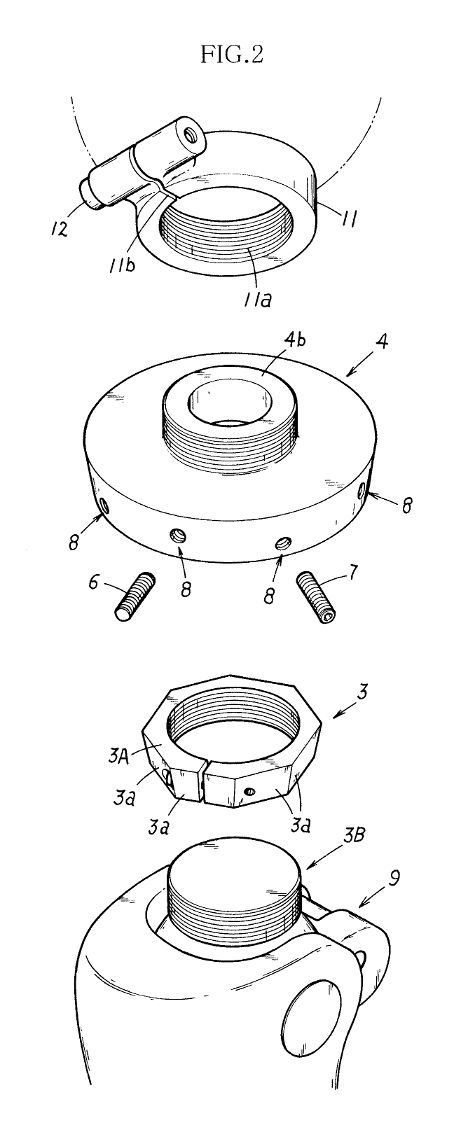

As shown in FIG. 2, the linking part 3 is formed by a member made of an appropriate metal, and is constituted by threaded attachment, in a mated state, of an annular body 3A to a lower linking part 3B of circular post shape projecting from the upper surface of the flexing part 9 provided to the lower prosthetic leg part 2.

This linking part 3 (the annular body 3A) is formed to octagonal shape in plan view, and is formed to have progressively greater diameter towards the upper end side, so that each of the eight outside peripheral surfaces constitutes a tapered surface 3a.

By providing these tapered surfaces 3a, when retained by the second retaining element 7 in the manner discussed below, the extent of planar abutment by the distal end surface of the second retaining element 7 is maximized, as compared with the case in which the outside peripheral surface of the linking part 3 is a simple vertical surface, making consistently satisfactory angular adjustments possible. Moreover, satisfactory retaining action of the linking part 3 is exhibited as well.

As shown in FIG. 2, the mated linking part 4 is a ceilinged cylindrical body formed of a member made of a suitable metal, and is constituted to be able to surround the linking part 3, discussed above, arranged within the mating aperture 4a.

A threaded linking part 4b onto which threads an upper linking part 11, discussed below, is formed on the upper surface of the top wall of the mated linking part 4.

Through-holes 8 composed of eight equally-spaced screw holes are formed in the mated linking part 4, the through-holes 8 being provided so as to open onto the inside peripheral surface of the mating aperture 4a and the outside peripheral surface of the mated linking part 4.

As shown in FIGS. 2, 3, and 6, the through-holes 8 are constituted by four first through-holes 8a provided in a direction orthogonal to the axis of the mated linking part 4, and four second through-holes 8b provided in a direction diagonal to the axis of the mated linking part 4, the first through-holes 8a and the second through-holes 8b being provided in alternating fashion in the circumferential direction of the mated linking part 4. The first through-holes 8a are provided at positions at the front, back, left, and right of the mated linking part 4 when the mated linking part 4 is viewed in the horizontal direction, and the second through-holes 8b are provided at positions between the first through-holes 8a.

The second through-holes 8b are provided in an inclined state such that the inside opening is at a higher position than the outside opening, and the angle of inclination of each of the second through-holes 8b is set so as to afford planar abutment of the distal end of the second retaining element 7, discussed later, against the tapered surface 3a of the linking part 3.

The retaining elements (the first retaining element 6 and the second retaining element 7) are threaded into the through-holes 8.

As shown in FIG. 2, the retaining elements are screw posts formed of members made of a suitable metal. With the linking part placed within the mating aperture 4a after threading the retaining elements into the mated linking part 4, the mated linking part 4 can be linked to the linking part 3 in this state, with the linking part 3 within the mating aperture 4a being retained by the distal ends of the retaining elements.

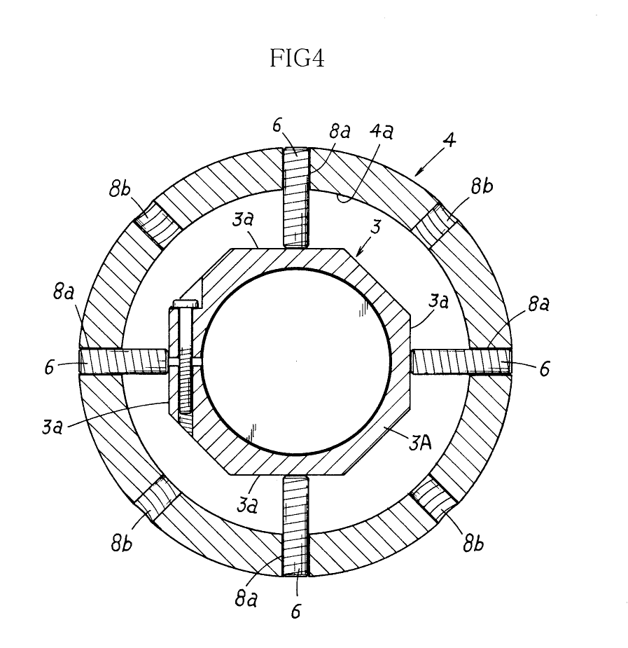

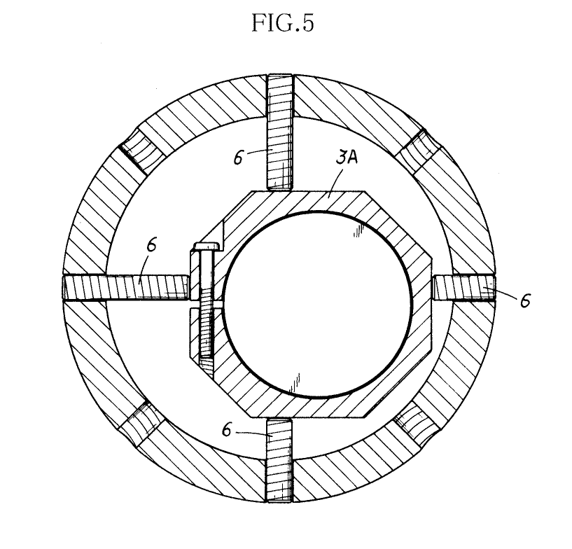

In specific terms, the first retaining elements 6 threaded into the first through-holes 8a protrude into the mating aperture 4a in directions orthogonal to the axis of the mated linking part 4 and retain the linking part 3, linking the mated linking part 4 to the linking part 3 through the agency of the first retaining elements 6, while allowing the linking part 3 to be moveably adjustable in the front-back and left-right directions with respect to the mated linking part 4, according to the degree of protrusion of each of the first retaining elements 6 (see FIGS. 4 and 5).

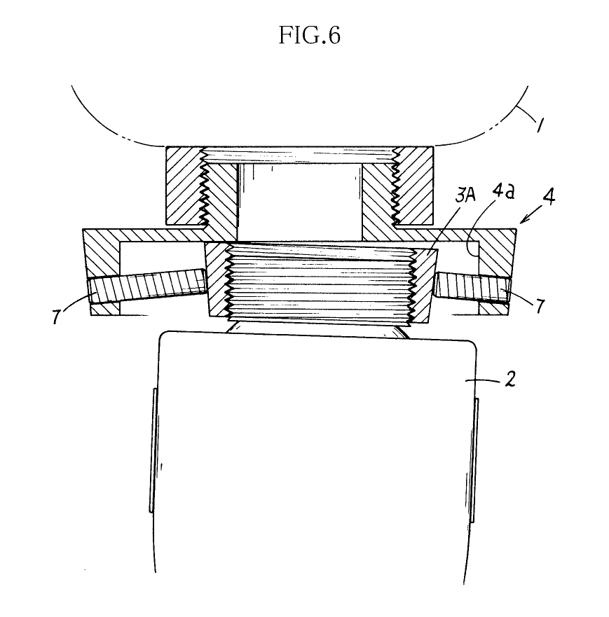

Meanwhile, a structure in which the second retaining elements threaded into the second through-holes 8b protrude into the mating aperture 4a obliquely relative to the axis of the mated linking part 4 and retain the linking part 3, linking the mated linking part 4 to the linking part 3 through the agency of the second retaining elements 7, produces angular adjustment functionality. The linking part 3 may be placed at an optimal angle in the mated linking part 4, for example, in such a way that the upper prosthetic leg part 1 and the lower prosthetic leg part 2 are linked at an optimal angle, in which state the lower prosthetic leg part 2 can be linked at a desired angle to the upper prosthetic leg part 1, by causing the second retaining elements 7 to protrude to an appropriate extent and retain the linking part 3, and/or by varying the degree of protrusion of the second retaining elements 7 to vary the angle of the linking part 3 with respect to the mated linking part 4 (see FIG. 6). When doing so, a proximal end part of the retaining element protruding from the outside opening of each of the through-holes 8 is cut off, the end being cut off in such a way that the cut surface is flush with the outside peripheral surface of the mated linking part 4.

The structure of the retaining elements is not limited to one of being threaded into the through-holes; retaining elements having a structure involving press-fit engagement would be acceptable. However, a detachable structure is preferred.

As shown in FIG. 2, the upper linking part 11 is an annular body formed by a member made of a suitable metal, and is provided in the lower part of the upper prosthetic leg part 1.

A thread groove 11a is formed in the internal aperture of the upper linking part 11, such that mated linking may be accomplished by threading to the mated linking part 4, discussed above.

Additionally, the upper linking part 11 has a slit part 11b formed at a prescribed location, allowing the diameter to be reduced, and a tightening member 12 for tightening the slit part 11b is provided between the opposed ends of the slit part 11b.

Consequently, after mated linking by threading the upper linking part 11 into the threaded linking part 4b of the mated linking part 4, the tightening member 12 is tightened, constricting the gap between the opposed ends of the slit part 11b, to obtain a state of secure linking of the upper linking part 11 to the mated linking part 4.

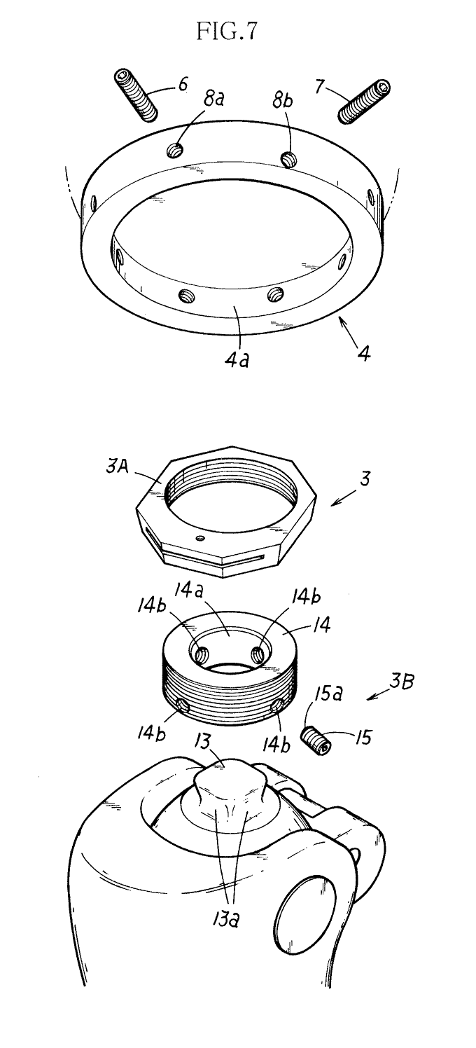

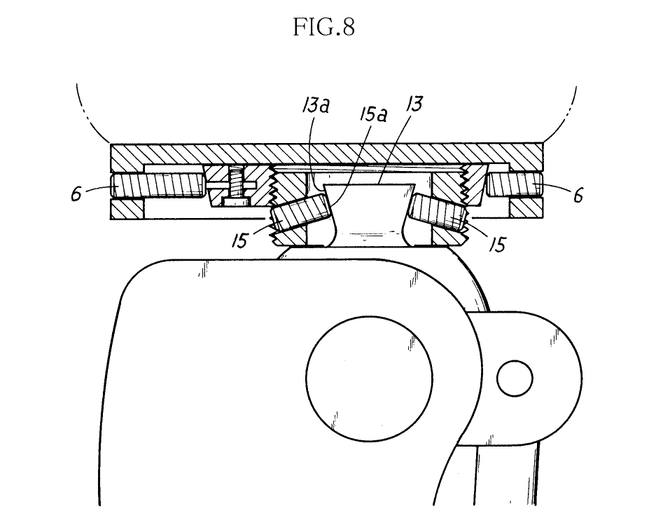

FIGS. 7 and 8 show another example of the lower linking part 3B constituting the linking part 3, this lower linking part 3B being constituted by a protruding part 13 provided to an upper part of the lower prosthetic leg part 2, and a mated linking member 14 linked to this protruding part 13.

As shown in FIG. 7, the protruding part 13 is an element of square post shape made of a suitable metal, projecting from the upper surface of a flexing part 7 provided to the lower prosthetic leg part 2.

This protruding part 13 is formed to have progressively greater diameter towards the upper end side, such that each of four outside peripheral surfaces constitutes a tapered surface 13a.

By providing these tapered surfaces 13a, when retained by a retaining element 15, discussed below, the extent of planar abutment by the distal end surface of the retaining element 15 is maximized, as compared with the case in which the outside peripheral surface of the protruding part 13 is a simple vertical surface, making consistently satisfactory angular adjustments possible. Moreover, satisfactory retaining action of the protruding part 13 is exhibited as well.

As shown in FIG. 7, the mated linking member 14 is formed of a member made of a suitable metal, and is constituted to be capable of surrounding the aforementioned protruding part 13 placed inside a mating aperture 14a.

A thread groove for threaded attachment of the annular body 3A is formed on the outside peripheral surface of the mated linking member 14, and through-holes 14b, discussed below, open onto the outside peripheral surface where the thread groove is formed.

Through-holes 14b composed of four equally-spaced screw holes are formed in the mated linking member 14, the through-holes 14b being provided so as to open onto the inside surface of the mating aperture 14a and the outside peripheral surface of the mated linking member 14.

As shown in FIG. 8, the through-holes 14b are provided in an inclined state such that the inside opening is at a higher position than the outside opening. The angle of inclination of each of the through-holes 14b is set to afford planar abutment of the distal end of the retaining element 15, discussed later, against the tapered surface 13a of the protruding part 13.

The retaining elements 15 are threaded into the through-holes 14b.

As shown in FIG. 7, the retaining elements 15 are screw posts formed of a member made of a suitable metal material. With the protruding part 13 placed in the mated linking member 14 after threading the retaining elements 15 into the mated linking member 14, the mated linking member 14 can be linked to the protruding part 13 in this state, with the protruding part 13 within the mating aperture 14a being retained by the distal ends of the retaining elements 15.

The structure whereby the mated linking member 14 is linked to the protruding part 13 through the agency of the retaining elements produces angular adjustment functionality, and with this constitution as well, the protruding part 13 may be placed at an optimal angle in the mated linking member 14, in such a way that the upper prosthetic leg part 1 and the lower prosthetic leg part 2 are linked at an optimal angle; and in this state, the lower prosthetic leg part 2 can be linked at a desired angle to the upper prosthetic leg part 1, by causing the retaining elements 15 to protrude to an appropriate extent and retain the protruding part 13 (see FIG. 8). When doing so, a proximal end part of the retaining element 15 protruding from the outside opening of each of the through-holes 14b is cut off, the end being cut off in such a way that the cut surface is flush with the outside peripheral surface of the mated linking member 14.

The structure of the retaining elements 15 is not limited to one of being threaded into the through-holes 14b; retaining elements having a structure involving press-fit engagement would be acceptable. However, a detachable structure is preferred.

According to the present embodiment, by virtue of being constituted in the manner shown above, when the upper prosthetic leg part 1 and the lower prosthetic leg part 2 are to be linked, the mated linking part 4 provided to the upper prosthetic leg part 1 is mated with the linking part 3 provided to the lower prosthetic leg part 2, and the linking part 3 within the mating aperture 4a is retained by the retaining elements 6, 7 which protrude into the mating aperture 4a of the mated linking member 4.

When the linking part 3 is retained by the retaining elements 6, 7, the retaining elements 6, 7 are caused to protrude to an appropriate extent and retain the linking part 3, in such a way as to produce a state of optimal fitting to the body (such that the upper prosthetic leg part 1 and the lower prosthetic leg part 2 are linked at an optimal angle).

In specific terms, the retaining elements 6, 7 are constituted by the first retaining element 6 which protrudes into the mating aperture 4a in a direction orthogonal to the axis of the mated linking part 4 and retains the linking part 3, and by the second retaining element 7 which protrudes into the mating aperture 4a in a direction diagonal to the axis of the mated linking part 4 and retains the linking part 3.

Due to this constitution, by appropriately varying the degree of protrusion of the first retaining element 6 into the mating aperture 4a, the first retaining element 6 protrudes in a direction orthogonal to the axis of the mated linking part 4 and presses the linking part 3, whereby the linking part 3 moves, in this case in a horizontal direction, within a plane orthogonal to the axis of the mated linking part 4 within the mating aperture 4a. Meanwhile, by appropriately varying the degree of protrusion of the second retaining element 7 into the mating aperture 4a, the second retaining element 7 protrudes obliquely relative to the axis of the mated linking part 4 within the mating aperture 4a and presses the linking part 3, whereby the linking part 3 tilts within the mating aperture 4a.

That is, for example, position adjustment of the lower prosthetic leg part 2 in a horizontal direction (e.g., the front-back direction or left-right direction) with respect to the upper prosthetic leg part 1 may be satisfactorily carried out merely by adjusting the degree of protrusion of the first retaining element 6, while angular adjustment of the lower prosthetic leg part 2 respect to the upper prosthetic leg part 1 may be satisfactorily carried out merely by adjusting the degree of protrusion of the second retaining element 7.

Consequently, according to the present embodiment, a state of optimal fit permitting satisfactory ambulation can be obtained easily, and moreover adjustments required by the user according to circumstances can be made easily and rapidly.

### Embodiment 2

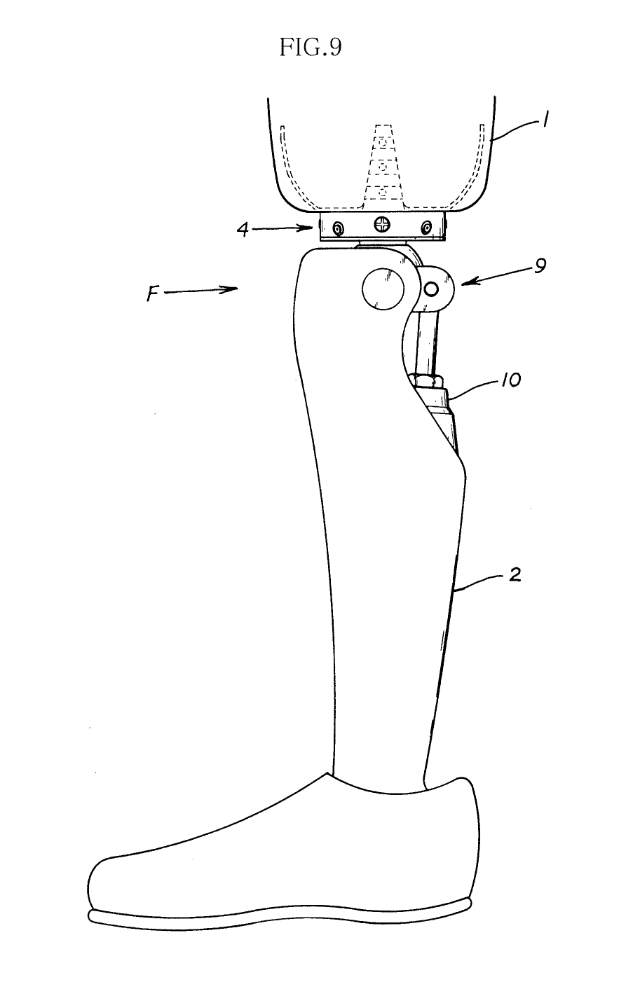

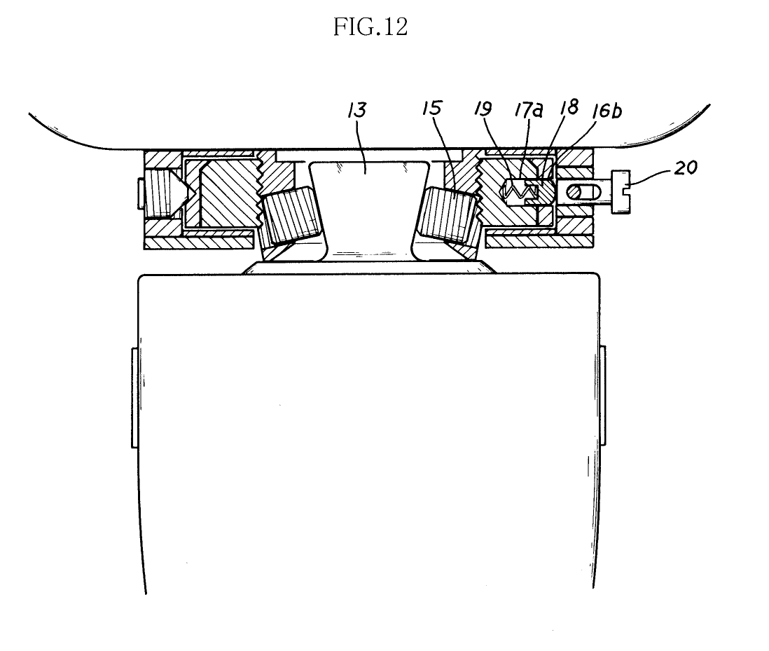

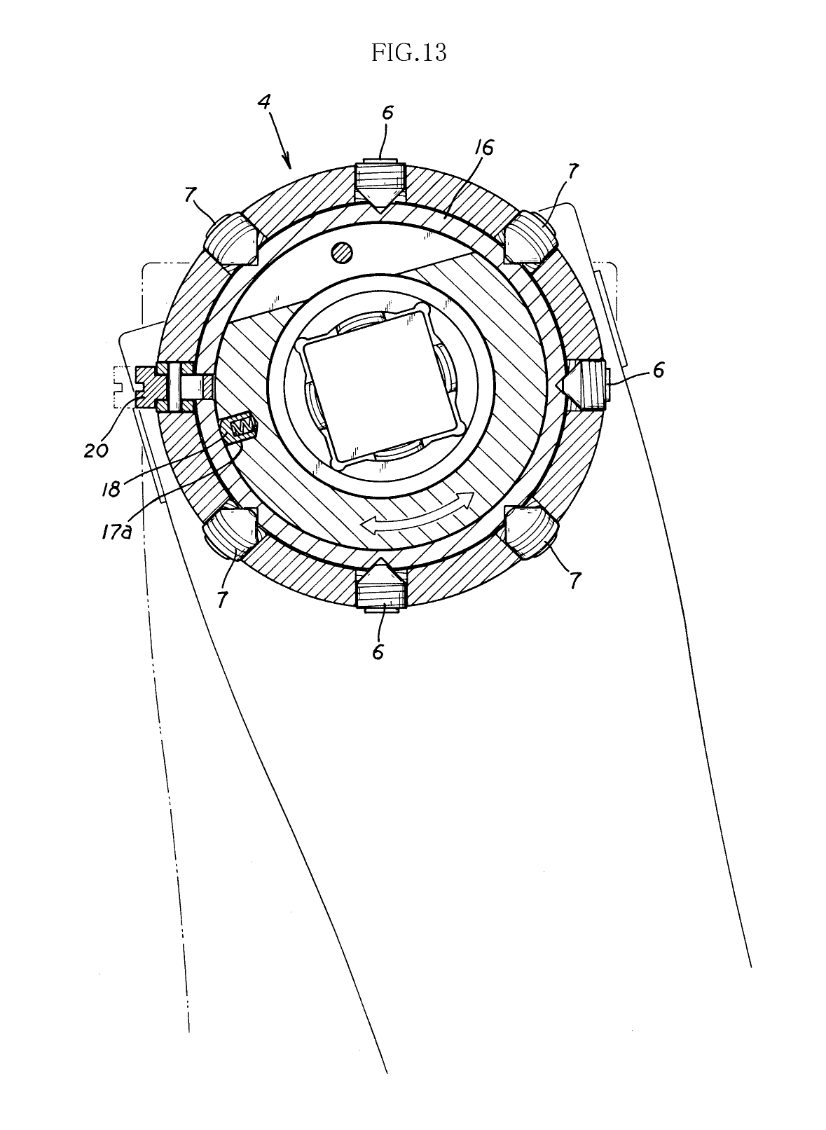

A specific second embodiment of the present invention will be described on the basis of FIGS. 9 to 15.

As shown in FIG. 9, in the present embodiment, the annular body 3A of the linking structure of the upper prosthetic leg part 1 and the lower prosthetic leg part 2 is equipped with a rotation mechanism, making the lower prosthetic leg part 2 rotatable with respect to the upper prosthetic leg part 1. This function is useful in situations such as sitting in a reception area or the like.

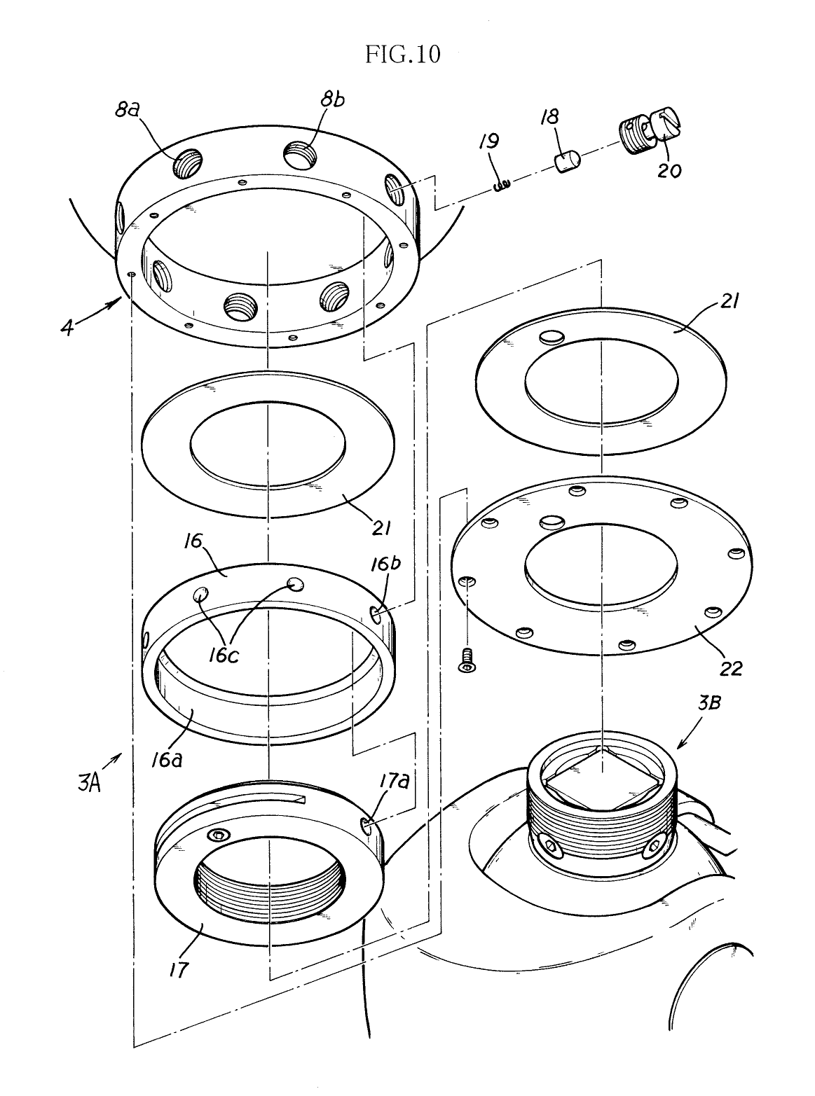

In specific terms, as shown in FIG. 10, the annular body 3A is formed by members made of a suitable metal, and is constituted by a first ring member 16, and a second ring member 17 which is positioned in axially rotatable fashion within a ring aperture 16a of the first ring member 16, with the axis thereof aligned with the first ring member 16.

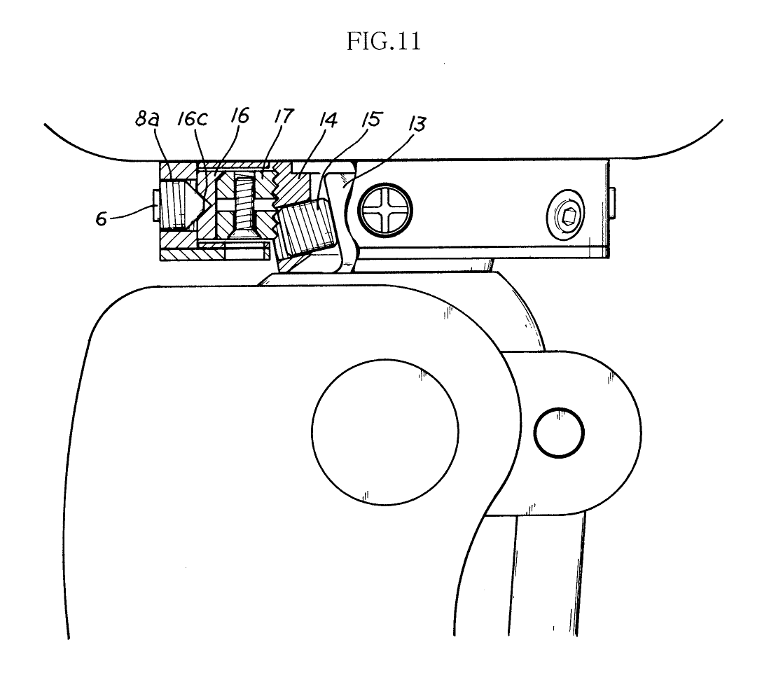

A through-hole 16b is provided at a prescribed location on the peripheral surface of this first ring member 16, and a recessed part 17a which aligns with this through-hole 16b is provided at a prescribed location on the peripheral surface of the second ring member 17. A slide member 18 is slidably provided between the through-hole 16b and the recessed part 17a. When this slide member 18 is in a state of bridging between the through-hole 16b and the recessed part 17a, axial rotation of the second ring member 17 with respect to the first ring member 16 is inhibited, and when the slide member 18 is pushed in opposition to a return urging body 19 by pushing a push button 20 provided to the mated linking part 4, the slide member 18 retracts into the recessed part 17, thereby releasing the second ring member 17 from a state of inhibited axial rotation with respect to the first ring member 16 (see FIGS. 11-13).

Consequently, by pushing the push button 20, the lower prosthetic leg part 2 becomes axially rotatable with respect to the upper prosthetic leg part 1.

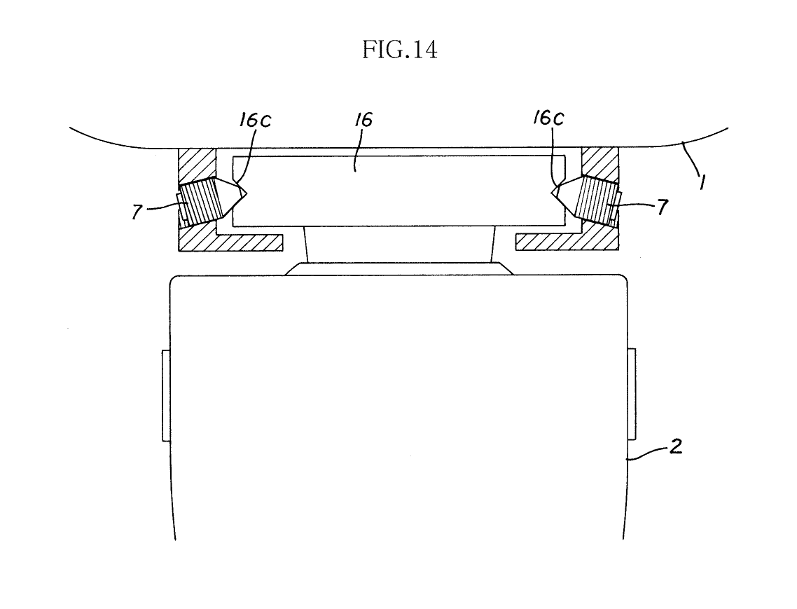

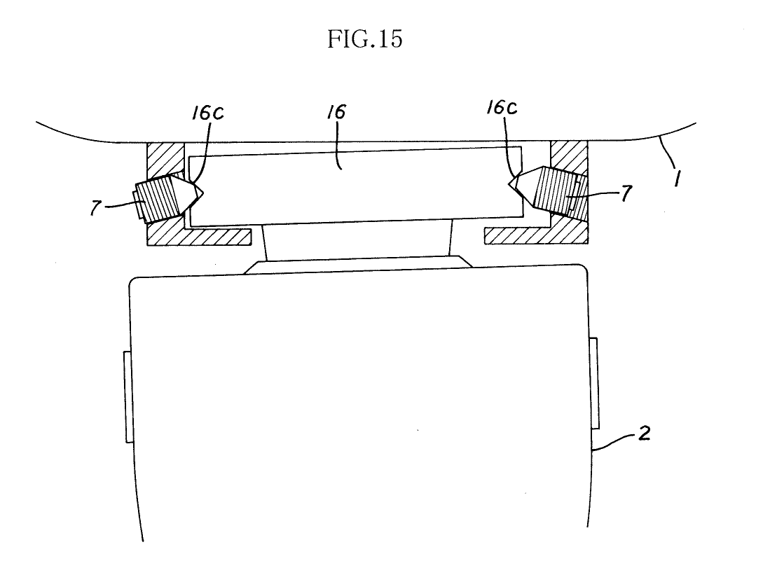

The outside peripheral surface of the first ring member 16 is provided with tapered recessed parts 16c, these tapered recessed parts 16c being designed for mortise-and-tenon engagement with pointed end parts provided to distal end parts of the retaining elements (the first retaining element 6 and the second retaining element 7) (see FIGS. 14, 15).

In the present embodiment, the lower linking part 3B is of the same type as that indicated in the other example of Embodiment 1 (see FIGS. 7, 8).

Symbol 21 indicates a spacer, and symbol 22 indicates a lid member.

The remaining features are the same as in Embodiment 1.

### Embodiment 3

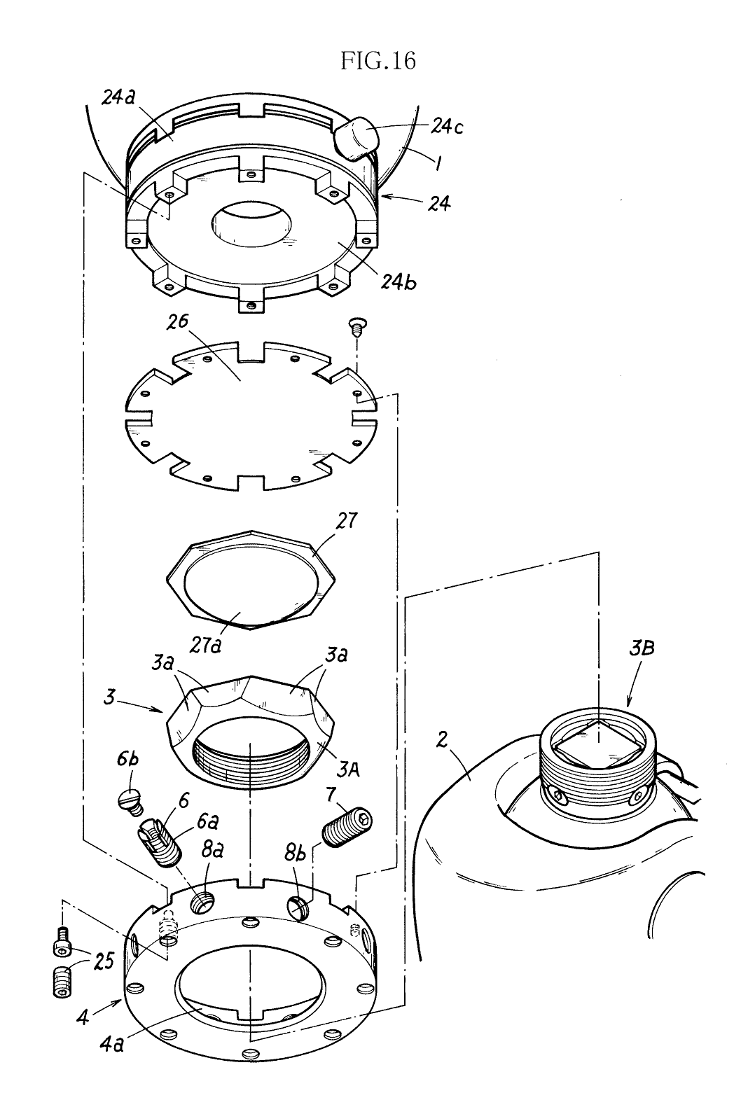

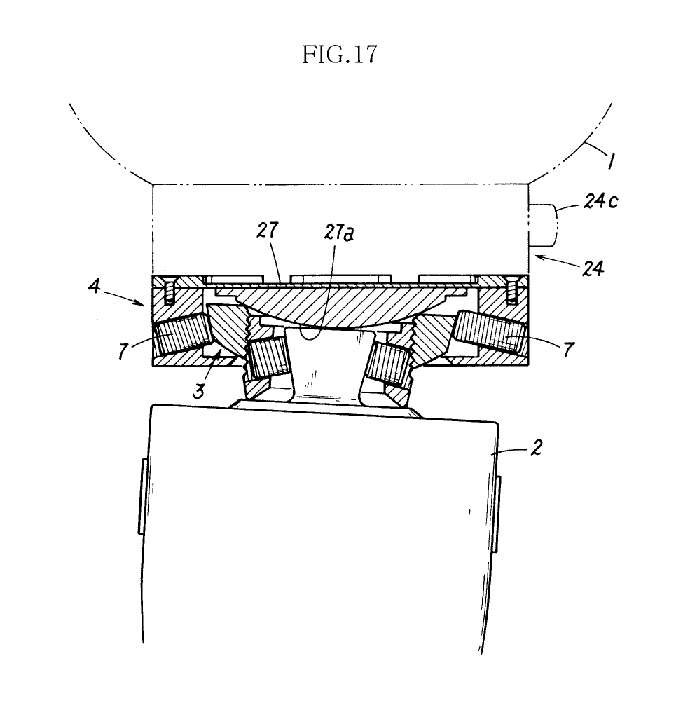

A specific third embodiment of the present invention will be described on the basis of FIGS. 16 and 17.

In this embodiment, a rotating device 24 is provided in the region linked to the lower prosthetic leg part 2 in the lower part of the upper prosthetic leg part 1, making the lower prosthetic leg part 2 rotatable with respect to the upper prosthetic leg part 1 through the agency of the rotating device 24.

In specific terms, as shown in FIG. 16, in this rotating device 24, a rotating part 24a is rotatably provided to a proximal part 24a provided to a lower part of the upper prosthetic leg part 1. This rotating part 24a is maintained in a state of inhibited rotation with respect to the proximal part 23a by a locking mechanism (not illustrated), and through pushing a push button 24c, is released from the state of inhibited rotation produced by the locking mechanism, allowing a rotating part 24b to rotate.

The mated linking part 4 for mated linking to the linking part 3 is detained in state of checked rotation to the rotating part 24b of this rotating device 24 by detaining members 25.

Consequently, in association with rotation of the rotating part 24b of the rotating device 24, the lower prosthetic leg part 2 axially rotates with respect to the upper prosthetic leg part 1 through rotation of the mated linking part 4.

Symbol 26 indicates a lid member for closing off an upper part of the mated linking part 4.

The present embodiment additionally has a support part for supporting a distal end part of the linking part 3, the region of the support part that supports the linking part 3 being provided with a convex curving surface.

In specific terms, as shown in FIG. 16, a support body 27 is provided at the distal end side (upper end side) of the annular body 3A of the linking part 3, the support body 27 being provided with a convex curving surface 27a which is positioned in an intermated state to the inside of the distal end-side opening of the annular body 3A, and which abuts the rim of the distal end-side opening.

Consequently, when the linking part 3 (the annular body 3A) is tilted with respect to the axis of the mated linking part 4, the annular body 3A is constantly kept in a state of support by this convex curving surface 27a (see FIG. 17), thereby maintaining a securely linked state.

In the present embodiment, as the first retaining element 6 there is adopted a threaded body equipped with a detachment-preventing function, having a detachment-preventing screw 6b which is threaded into the proximal end part of a main body part 6a in which a screw groove has been formed on the peripheral surface.

This threaded body equipped with a detachment-preventing function has a plurality of slits formed in a lengthwise direction in the proximal end part of the main body part 6a into which the detachment-preventing screw 6b is threaded, allowing the proximal end part of the main body part 6a to expand in diameter when the detachment-preventing screw 6b is threaded therein, thereby producing a detachment-preventing function. This threaded body equipped with a detachment-preventing function may be adopted as the second retaining element 7 as well.

The remaining features are the same as in Embodiment 1.

The present invention is not limited to Embodiments 1-3, and the specific constitutions of the constituent elements may be designed as appropriate.

1-13. (canceled)

14. A structure for linking an upper prosthetic leg part fitted to the body, and a lower prosthetic leg part linked to the upper prosthetic leg part to constitute a prosthetic leg, a ground-contacting part being provided at a lower end, the structure for linking an upper prosthetic leg part and a lower prosthetic leg part in a prosthetic leg being characterized in being constituted by a linking part provided to either the lower prosthetic leg part or the upper prosthetic leg part, and a mated linking part provided to the other of the lower prosthetic leg part and the upper prosthetic leg part and adapted to mate with the linking part; there being provided retaining elements which protrude into the mating aperture of the mated linking part and retain the linking part positioned within the mating aperture, the retaining elements being constituted by a first retaining element which protrudes into the mating aperture in a direction orthogonal to the axis of the mated linking part and retains the linking part, and a second retaining element which protrudes into the mating aperture in a direction diagonal to the axis of the mated linking part and retains the linking part; the degree of protrusion of the first retaining element into the mating aperture being variable, and the linking part being moveably linked within a plane orthogonal to the axis of the mated linking part within the mating aperture.

15. The structure for linking an upper prosthetic leg part and a lower prosthetic leg part in a prosthetic leg according to claim 14, wherein the structure for linking an upper prosthetic leg part and a lower prosthetic leg part in a prosthetic leg is characterized in that the degree of protrusion of the second retaining element into the mating aperture is variable, and the linking part is tiltably linked within the mating aperture.

16. The structure for linking an upper prosthetic leg part and a lower prosthetic leg part in a prosthetic leg according to claim 14, wherein the structure for linking an upper prosthetic leg part and a lower prosthetic leg part in a prosthetic leg is characterized in that the mated linking part is provided with a plurality of through-holes, the first retaining element and the second retaining element being inserted into each of the through-holes.

17. The structure for linking an upper prosthetic leg part and a lower prosthetic leg part in a prosthetic leg according to claim 15, wherein the structure for linking an upper prosthetic leg part and a lower prosthetic leg part in a prosthetic leg is characterized in that the mated linking part is provided with a plurality of through-holes, the first retaining element and the second retaining element being inserted into each of the through-holes.

18. The structure for linking an upper prosthetic leg part and a lower prosthetic leg part in a prosthetic leg according to claim 16, wherein the structure for linking an upper prosthetic leg part and a lower prosthetic leg part in a prosthetic leg is characterized in that the through-hole into which the second retaining element inserts is provided obliquely relative to the axis of the mated linking part.

19. The structure for linking an upper prosthetic leg part and a lower prosthetic leg part in a prosthetic leg according to claim 17, wherein the structure for linking an upper prosthetic leg part and a lower prosthetic leg part in a prosthetic leg is characterized in that the through-hole into which the second retaining element inserts is provided obliquely relative to the axis of the mated linking part.

20. The structure for linking an upper prosthetic leg part and a lower prosthetic leg part in a prosthetic leg according to any of claims 14-19, wherein the structure for linking an upper prosthetic leg part and a lower prosthetic leg part in a prosthetic leg is characterized in that an outside peripheral surface of the linking part defines an inclined surface that widens to the distal end side.

21. A structure for linking an upper prosthetic leg part fitted to the body, and a lower prosthetic leg part linked to the upper prosthetic leg part to constitute a prosthetic leg, a ground-contacting part being provided at a lower end, the structure for linking an upper prosthetic leg part and a lower prosthetic leg part in a prosthetic leg being characterized in being constituted by a linking part provided to either the lower prosthetic leg part or the upper prosthetic leg part, and a mated linking part provided to the other of the lower prosthetic leg part and the upper prosthetic leg part and adapted to mate with the linking part; there being provided retaining elements which protrude into the mating aperture of the mated linking part and retain the linking part positioned within the mating aperture, the retaining elements being constituted by a first retaining element which protrudes into the mating aperture in a direction orthogonal to the axis of the mated linking part and retains the linking part, and a second retaining element which protrudes into the mating aperture obliquely relative to the axis of the mated linking part and retains the linking part; the mated linking part being provided with a plurality of through-holes, and the first retaining element and the second retaining element being inserted into each of the through-holes.

22. The structure for linking an upper prosthetic leg part and a lower prosthetic leg part in a prosthetic leg according to claim 21, wherein the structure for linking an upper prosthetic leg part and a lower prosthetic leg part in a prosthetic leg is characterized in that the through-hole into which the second retaining element inserts is provided obliquely relative to the axis of the mated linking part.

23. The structure for linking an upper prosthetic leg part and a lower prosthetic leg part in a prosthetic leg according to claim 21 or 22, wherein the structure for linking an upper prosthetic leg part and a lower prosthetic leg part in a prosthetic leg is characterized in that the degree of protrusion of the second retaining element into the mating aperture is variable, and the linking part is tiltably linked within the mating aperture.

24. A structure for linking an upper prosthetic leg part fitted to the body, and a lower prosthetic leg part linked to the upper prosthetic leg part to constitute a prosthetic leg, a ground-contacting part being provided at a lower end, the structure for linking an upper prosthetic leg part and a lower prosthetic leg part in a prosthetic leg being characterized in being constituted by a linking part provided to either the lower prosthetic leg part or the upper prosthetic leg part, and a mated linking part provided to the other of the lower prosthetic leg part and the upper prosthetic leg part and adapted to mate with the linking part; there being provided retaining elements which protrude into the mating aperture of the mated linking part and retain the linking part positioned within the mating aperture, the retaining elements being constituted by a first retaining element which protrudes into the mating aperture in a direction orthogonal to the axis of the mated linking part and retains the linking part, and a second retaining element which protrudes into the mating aperture obliquely relative to the axis of the mated linking part and retains the linking part; and an outside peripheral surface of the linking part defines an inclined surface that widens to the distal end side.
